(19) United States
(12) Patent Application Publication
(10) Pub. No.: US 2019/0118034 A1
(43) Pub. Date: Apr. 25, 2019

(54) A SYSTEM AND A METHOD FOR CONVERTING EXERCISE ACTIVITY INTO USER CREDIT

(71) Applicant: Fitness Village Oy, Oulu (FI)

(72) Inventors: Jouni HERRANEN, Oulunsalo (FI); Jari JÄRVINEN, Jyväskylä (FI)

(21) Appl. No.: 16/090,417

(22) PCT Filed: Apr. 6, 2017
(86) PCT No.: PCT/FI2017/050246

§ 371 Date: Oct. 1, 2018

(30) Foreign Application Priority Data
Apr. 6, 2016 (FI) 20165297

(51) Int. Cl. A63B 24/00; G06Q 30/06; H04L 29/06; A61B 5/11; A61B 5/00

(52) U.S. Cl. CPC A63B 24/0059; G06Q 30/0619; H04L 63/10; A63B 24/0084; A63B 2225/20; A61B 5/681; A61B 5/6898; A63B 2024/0068; A61B 5/1118

(57) ABSTRACT

A system for converting exercise activity into user credit in a networked multi-user virtual environment, the system including one or more networked server(s) arranged to run the multi-user virtual environment, wherein the system is arranged to obtain from a networked third party system information including user identification for a user and user's exercise activity, which information which information is protected data and has been stored in the third party system and wherein the networked multi-user virtual environment has been authorized to access the data, convert the user's exercise activity into credit for the user in the networked multi-user virtual environment, and store the user credit in the networked multi-user virtual environment. The present application also relates to a method for converting exercise activity into user credit in a networked multi-user virtual environment.

### FIELD OF THE APPLICATION

The present application relates to a system for converting exercise activity into user credit in a networked multi-user virtual environment. The present application also relates to a method for converting exercise activity into user credit in the networked multi-user virtual environment.

### BACKGROUND

A variety of exercise equipment are arranged to monitor the exercise performance of persons using said equipment. In many cases the equipment monitor or calculate exercise parameters, such as calories burned, exercise time, resistance, load, distance etc. These parameters and results may be shown to the person exercising i.e. the user, and they may be stored on the exercise equipment. Such exercise equipment are used in for example gyms, fitness centers, health clubs and the like, and may include for example stationary bicycles, treadmills, elliptical machines, rowing machines, and the like.

The results obtained from an exercise are usually merely informative for the user. The user may monitor the progress of the exercise or the development of his or her physical condition or fitness. The equipment provide very little or not at all further motivation for the user to carry on exercising or to boost the exercise.

Further, the obtained results are not comparable to results from different exercises or from different devices. It has been very challenging to compare results from persons with different exercise background, such as an advanced exerciser and a beginner.

### SUMMARY

One embodiment provides a system for converting exercise activity into user credit in a networked multi-user virtual environment, the system comprising one or more networked server(s) arranged to
run the multi-user virtual environment, wherein the system is arranged to
obtain from a networked third party system information comprising user identification for a user and the user's exercise activity, which information is protected data and has been stored in the third party system and wherein the networked multi-user virtual environment has been authorized to access the data,
convert the user's exercise activity into credit for the user in the networked multi-user virtual environment, and
store the user credit in the networked multi-user virtual environment.

One embodiment provides a method for converting exercise activity into user credit in a networked multi-user virtual environment, the method comprising
providing the system arranged to run the multi-user virtual environment,
obtaining from a networked third party system information comprising user identification for a user and the user's exercise activity per day, which information is protected data and has been stored in the third party system and wherein the networked multi-user virtual environment has been authorized to access the data,
converting the user's exercise activity into credit for the user, and
storing the user credit in the networked multi-user virtual environment.

One embodiment provides a computer-readable data storage medium having a computer-executable program code stored operative to perform the method.

The main embodiments are characterized in the independent claims. Various embodiments are disclosed in the dependent claims. The embodiments recited in dependent claims and in the specification are mutually freely combinable unless otherwise explicitly stated.

With the method and algorithm presented in the present application it is possible to convert different types of exercises into user credit and to obtain comparable credits.

As the method is operated in a multi-user environment it is possible to motivate a single user to exercise in a community, wherein the exercise activities of all the users have an effect to the limits and incentives set by the system. When the performance of a single user is compared to the performances of other individuals, it provides high motivation for a single user to perform better, for example to exercise more. It is also possible to reliably compare the exercise activities of exercisers with different backgrounds, especially in the community.

The exercise data and the user data is imported from an existing database, so the user does not have to enter or transfer the data.

### BRIEF DESCRIPTION OF THE FIGURES

FIG. 1 shows a flowchart of an example of the arrangement wherein data from different sources is transferred to a third party system and further to the virtual environment, wherein it is converted into virtual credit, which may be used in the environment or outside the environment.

FIG. 2 shows an application of a generic natural logarithm y=ln(x) for determining the credits (y-axis) derived from the consumed calories (x-axis), with upper and lower limits. The valid zone is hatched.

FIG. 3 shows an example of credits calculated with the algorithm of an embodiment (y-axis) from the daily calories (x-axis), wherein daily credit MAX is 300 kcal, which is the average consumed calories of all users.

FIG. 4 shows a flowchart of an example of the process of converting exercise into a deposit of credit in a virtual system account.

FIG. 5 shows a detailed chart of the interactions between different systems in an example of the process

### DETAILED DESCRIPTION

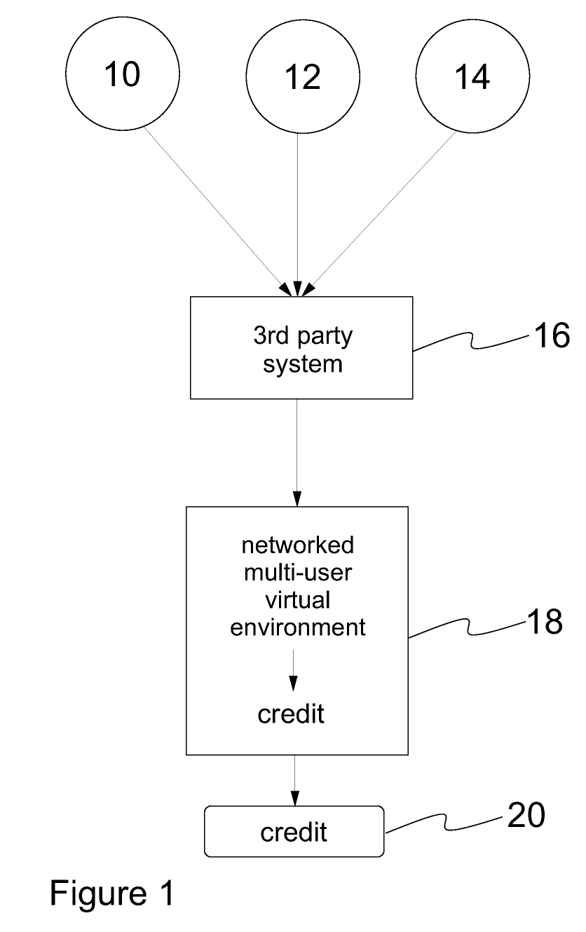

One embodiment provides a system for converting user's exercise activity into user credit in a networked multi-user virtual environment 18 as shown in the example of FIG. 1. A networked virtual environment refers to an information system which is accessible via information network, such as Internet. The virtual environment is run in a system comprising one or more servers, which are connected to the network. A user may log in to the system and carry out activities provided by the system in the virtual environment. A user usually has an user account in the system, preferably secured with a password. The user account may be created by when the user first time enters the system and registers to the system. The user account may be alternatively created in a third party system and transferred or replicated to the virtual environment. The user is provided with personal settings. The user account, and the environment related to it, is personalizable so the user may change the settings and the environment, and the user's actions may have an effect to the user's personal environment. On or more of the actions described herein, or all of them, may be carried out automatically in the networked multi-user virtual environment.

The virtual environment 18 is a multi-user virtual environment, which means that the environment is arranged to host multiple users, at least two, but even hundreds or thousands of users substantially simultaneously. The users may form a community, such as a virtual community in the virtual environment. Each user has a personal user account and personal settings, in practice personal environment in the system. The users may interact with each other and see the actions of the other users. A user may be represented by an avatar and/or a nickname, which may be chosen by the user. The user may also be considered or called as a participant, a player, a virtual citizen, a visitor or the like in the environment. The exercise activities of each user are gathered to the system, and the collective exercise activity of all the users has an effect to the system, for example to the way the activities are accepted and/or converted and how the users are motivated to exercise.

The one or more servers contain one or more software(s) installed and arranged to carry out the required actions to run the virtual environment and perform the method described herein. More particularly the system comprising one or more networked server(s) is arranged to run the multi-user virtual environment, and to carry out any of the method steps described herein.

The virtual environment may be an educational virtual environment, a game-like virtual environment, such as a game or other environment with game-like elements, a social media in a form of a virtual environment, or a combination thereof. If the virtual environment is a game or game-like virtual environment, the conversion of exercise activity into the user credit is not necessarily part of the game itself.

The system is arranged to obtain from a networked third party system 16 information comprising user identification for a user and user's exercise activity, which information has been stored in the third party system.

A networked third party system 16 is a system run by a party which usually is not the party running the networked multi-user virtual environment described herein. The third party may be for example an exercise equipment provider, an information system provider, a personal device provider, or a manufacturer thereof, or any other commercial party or the like. The third party system is a networked system comprising one or more servers arranged to obtain information from one or more networked devices and/or from a user. This information is related to exercise events carried out by an individual person, more particularly the user. The information comprises at least information comprising user identification for a user and information comprising user's personal exercise activity. The user has a personal user account in the third party system, and the personal data obtained and collected in the system is associated with the user account. The third party system contains a database, wherein the user information, preferably multiple user information, is stored and wherefrom the information may be obtained. The data in the third party system is usually protected data, wherein the virtual environment has to be authorized to access the data. The third party system may be separated from the networked multi-user virtual environment by a network boundary protection. The networked multi-user virtual environment is arranged to request the data or the information from the third party system. The information comprising user identification for a user and the user's exercise activity per day are owned by the user, i.e. they are not owned by the third party system.

One example of such third party system is a system provided by an exercise device manufacturer, seller or a party related to the manufacturer or seller. A user exercising on an exercise unit or equipment is identified and the exercise data is collected and sent to the third party system, wherein it is associated with the user account and stored. The exercise unit is networked, for example with a wired or with a wireless network connection, and arranged to connect to a networked third party system. The third party system may be arranged to obtain and collect information of plurality of exercise units 10, 12, 14, which may be similar or different, for plurality of users. For example for a user information from more than one exercise units and/or exercise events 10, 12, 14 may be obtained and sent to the third party system 16 during the same exercise event, such as during a visit to a gym, a fitness center or a health club, wherein the user may utilize several exercise machines or units during the workout.

Another example of a third party system is a system provided by a manufacturer or a seller of personal devices, such as biometric monitors, mobile terminals, such as smartphones, tablets; wearable devices or the like. Such devices are usually not exercise units as such, but they may detect and/or collect information relating to the user, to users activity and to user's vital functions, such as heart rate. The personal devices are networked, usually wireless devices, which are arranged to connect to a networked third party system. In one embodiment a personal device is a heart rate monitor, which may also contain other types of biometric monitor functions. In one example a personal device comprises a pedometer. A personal device may be arranged to detect, monitor and/or calculate exercise information from the user or the user's activity, such as burned calories.

Examples of the personal devices include miCoach devise, Bluetooth Smart HRM, Android Wear, Armour39, Polar Heart Rate Strap, Runtastic Combo HRM, Smart Body Analyser, scales etc. These are also devices with are compatible with certain tracking platform systems, such as Google Fit.

Still another example of a third party system is a system provided by an information system provider, such as a user exercise tracking platform. Such a system may be arranged to obtain user exercise information for multiple sources, such as from applications and devices. The information may be combined and stored in the system. Such a third party system is not bound to a specific equipment or device, or to a specific brand of devices, but versatile information related to user exercise may be obtained and combined. One example of such a third party system is Google Fit, which is a health-tracking platform developed by Google for the Android operating system. It is a single set of APIs that blends data from multiple apps and devices. Google Fit uses sensors in a user's activity tracker or mobile device to record physical fitness activities (such as walking or cycling), which are measured against the user's fitness goals to provide a comprehensive view of their fitness. Confirmed partners include Nike, HTC, LG, Withings, Motorola, Noom, Runtastic, RunKeeper and Polar. Users can choose who their fitness data is shared with as well as delete this information at any time. A number of apps are integrated with Google Fit. Some examples are Aqualert, Nike, Polar Beat, Running, and Strava. Another example of such third party system is Apple's Health, which is intended to be a personal and central data collection point, for connected third-party electronic accessories and wearable technology that can directly monitor and analyze an individual's biochemistry and physiology for medical and general fitness purposes

After the information comprising user identification for a user and user's exercise activity stored in the third party system 16 is obtained from the third party system to the networked multi-user virtual environment 18 of the embodiments, the exercise activity must be converted into a form which is useful in the virtual environment and wherein different types of activities are comparable.

The networked virtual environment system is configured to obtain the information from the third party system by using any suitable protocol. One example of such concept is OAuth protocol, such as OAuth 2. This protocol allows third-party applications to grant limited access to an HTTP service, either on behalf of a resource owner or by allowing the third-party application to obtain access on its own behalf. Access is requested by a client, it can be a website or a mobile application for example. Oauth2 defines four roles: Resource Owner, Resource Server, Client, and Authorization Server.

Resource Owner is an entity capable of granting access to a protected resource. When the resource owner is a person, it is referred to as an end-user. Resource Owner is generally the user in the virtual environment

Resource Server is a server hosting protected data, more particularly a server hosting the protected resources and capable of accepting and responding to protected resource requests using access tokens (for example Google hosting the user's profile and personal information).

Client is an application requesting access to a resource server, more particularly an application making protected resource requests on behalf of the resource owner and with its authorization (for example a PHP website, a Javascript application or a mobile application). The term “client” does not imply any particular implementation characteristics (e.g. whether the application executes on a server, a desktop, or other devices).

Authorization Server is a server issuing access token to the client after successfully authenticating the resource owner and obtaining authorization. This token will be used for the client to request the resource server. The authorization server may be the same server as the resource server or a separate entity. In one example the Authorization Server is the same as the Resource Server. A single authorization server may issue access tokens accepted by multiple resource servers. The Resource Server and the Authorization Server are third party systems, which are contacted by the Client. The Client may be an application, a server, or both.

Tokens are random strings generated by the authorization server and are issued when the client requests them. There are two types of tokens: Access Token and Refresh Token. Access Token is the most important because it allows the user data from being accessed by a third-party application. This token is sent by the client as a parameter or as a header in the request to the resource server. It has a limited lifetime, which is defined by the authorization server. It must be kept confidential as soon as possible but this is not always possible, especially when the client is a web browser that sends requests to the resource server via Javascript. Refresh Token is a token issued with the access token but unlike the latter, it is not sent in each request from the client to the resource server. It merely serves to be sent to the authorization server for renewing the access token when it has expired. For security reasons, it is not always possible to obtain this token.

OAuth2 usually requires the use of HTTPS for communication between the client and the authorization server because of sensitive data passing between the two (tokens and possibly resource owner credentials). In general the communication between the virtual environment and the third party system are secured, for example encrypted, for example by using HTTPS.

The networked multi-user virtual environment is arranged to convert the user's exercise activity into credit for the user and store this user credit, which may be virtual credit, in the virtual environment. This is carried out by using one or more algorithms which are implemented as one or more computer-executable program code stored operative to perform the algorithm, or more particularly the method described herein, to obtain a user credit value as a result of the conversion. The program code(s) may be run or executed in the one or more servers.

The exercise may refer to any physical exercise or activity wherein it is possible to measure, calculate or otherwise determine the amount of the work done during a time period. The exercise may be exercise carried out in a gym, fitness center, health club or the like, such as weight training using free weights and/or exercise machines, aerobic exercises, for example by using exercise bicycle, threadmill, elliptical trainer, cross trainers and the like, or aerobic exercises carried out by walking, running, cycling, rollerskating, rollerblading, skating, skiing, rowing, and the like, for example outdoor activities. Also playing games, such as ball games, may be considered aerobic exercises. When using an exercise device the device may be arranged to determine and provide activity information, such as to provide consumed calories, repetitions, steps, resistance, exercise weight, exercise time and the like. During aerobic exercises, such as during outdoor exercises, group exercises at the gym and the like, especially when no exercise devices are used, it is possible to use personal devices, such as biometric monitors, mobile terminals and the like, which are capable of determining and outputting the exercise activity.

The user exercise activity may be provided in a variety of units or forms. In one embodiment user exercise activity is provided as calories (cal), or kilocalories (kcal). In such case the conversion of the measurements from the exercise event into a numerical value is usually carried out already in an exercise equipment or in a personal device. In one embodiment user exercise activity is provided as steps. In one embodiment user exercise activity is provided as activity time, for example as second, minutes, hours, or as a combination thereof. In many cases the information stored in the third party system is provided in calories. However, it is possible to convert the information obtained from the third party system into a different form before the conversion, for example all the versatile data combined from different sources and transferred to the virtual environment is converted into one form, such as into calories, in the virtual environment before the conversion.

Also other information associated with the user may be obtained from the networked third party system. This information may relate to the exercise, to the user, to exercise conditions, to location, to time or to season, or to any other relevant matter, such as nutrition, for example consumed calories and/or macronutrient ratio. Examples of exercise-related information include the type or name of the exercise, distance, weight, reps, speed, distance delta, map coordinates, steps and the like. Examples of user-related information include height, weight, heart rate, BMR, and body fat percentage. Time information may include duration of one or more workout, date of the exercise, and the like, for example in a form of one or more time stamps. Information may include geographical data or information, for example obtained from a personal device having GPS functionalities, such as GIS (geographic information system) coordinates. Locations or extents in Earth space-time may be recorded as dates/times of occurrence, and x, y, and z coordinates representing, longitude, latitude, and elevation, respectively.

In one embodiment cumulative user's exercise activity in exercise activity units per day is compared to a lower limit and/or to a higher limit for a day, wherein the user's exercise activity per day in exercise activity units must be higher than the lower limit and/or lower than the higher limit in order to be accepted and converted into the user credit. The user credit is personal and it is deposited into user account in the system. There may be low and/or high limits for daily deposit and/or accepted exercise.

One way to control the accumulation and/or deposit of credit is to consider the exercise on daily basis. This is advantageous especially in cases wherein it is desired to encourage the user to exercise a certain optimal amount per day. For example too much and/or too little exercise per day does not yield as good credit accumulation as an optimal amount of exercise per day does. Therefore one variable which may be used in the present embodiments is user's exercise activity per day in exercise activity units (unitsᵤₛₑᵣ). Day refers to a 24 hour period, for example from 0:00 AM to 0:00 PM, or 0:00 to 24:00. In one embodiment the activity units are calories. Usually kilocalories are used as basic units. Other types of exercise activity units may be converted into calories or kilocalories.

Another variable which may be used in the present embodiments is average exercise activity units of all users per day (units_{average}). All users refer to the number all users of the virtual environment, such as all registered users, or all active users, especially all active users on the current day. Active user may refer for example to a user who has exercise activity units per the particular day, or who has obtained credit for the particular day, or for a particular time period, such as 2, 3, 4, 5, 6 or 7 days, or 1, 2, 3, or 4 weeks, to exclude the effect of inactive users in the system. This may be calculated by dividing the sum of obtained exercise activity units of all users per day (unitsₐₗₗ) by the number of users. When the average exercise activity units of all users per day is calculated, the performance of a single user may be compared to the average performance. This enables presenting incentives for the single user to exercise.

In one embodiment converting the user's exercise activity per day into credit comprises
providing the user's exercise activity per day in exercise activity units (unitsᵤₛₑᵣ), such as calories,
calculating average exercise activity units of all users per day (units_{average}) in the virtual environment by dividing the sum of obtained exercise activity units of all users per day (unitsₐₗₗ) by the number of the users,
comparing the user's exercise activity units per day (unitsᵤₛₑᵣ) with the average exercise activity units of all users per day (units_{average}), wherein
if the user's exercise activity units per day is higher or equal compared to the average exercise activity units of all users per day, the user gets higher amount of credit than if the user's exercise activity units per day is lower compared to the average exercise activity units of all users per day.

In one embodiment comparing the user's exercise activity units per day (unitsᵤₛₑᵣ) with the average exercise activity units of all users per day (units_{average}) is carried out by comparing natural logarithms of the user's exercise activity units per day (ln(unitsᵤₛₑᵣ)) and the average exercise activity units of all users per day (ln(units_{average})).

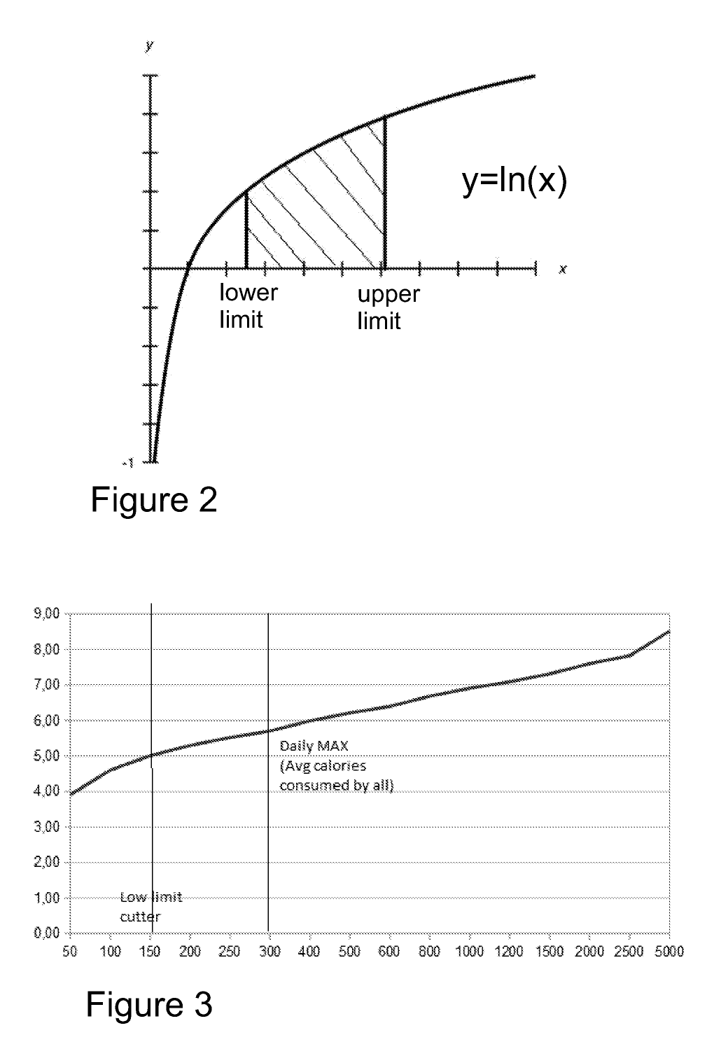

The natural logarithm of a number is its logarithm to the base of the mathematical constant e. Natural logarithms are used in the calculation of the credit to obtain comparative results. When activities of different people from different types of exercises are compared, it was found out that when using natural logarithms for converting the user's activity units, it was possible to obtain results which were divided evenly in the desired credit range. FIG. 2 illustrates the effect of ln to the result. The coefficients on the natural logarithm scale are directly interpretable as approximate proportional differences. Therefore different activity units, for example from different sources, from different exercisers or from different exercises, may be used and combined to obtain proportional credits. For example it is possible to compare the performances of an experienced marathon type runner and a beginner doing short walks, and to encourage both to exercise more, based on their personal levels.

An intermediate variable X is defined. In one embodiment the comparison is carried out with the following formula:

if ln(unitsᵤₛₑᵣ)<ln(units_{average}) then X=ln(unitsᵤₛₑᵣ),

else X=ln(units_{average})+n,

for example wherein the n has a value in the range of 0-0.5, preferably 0.1-0.5, wherein X is used for determining the user credit for the current day. The current day refers to a specific day of interest for which the calculation or comparison is carried out, for example day Z.

The value n is a correction value which may be used as in incentive or a bonus for a user who exercises more than an average user. For example if a user consumes more calories per day than all the users in average, the user is awarded with a small bonus, which in a long run will raise the average of all the users thereby motivating all the users to exercise more. However, the average exercise activity units of all users per day will rise excessively even if certain users exercise substantially more than average users, such as several hours per days. Even if a user consumes less calories than all the users in average, the user will be awarded accordingly, but however the user is motivated to exercise at least the minimum amount per day. The n is proportional to the level of user's exercise activity, wherein a higher level or amount of exercise, such as user's exercise activity units per day or per another time period, such as 2, 3, 4, 5, 6 or 7 days, or 1, 2, 3, or 4 weeks, will provide a higher value of n. The n may have a value of zero if the performance of the user does not fulfil predetermined requirements, and it may be more than zero if the user performs better than average or a predetermined level. In such case n may be for example at least 0.05 or 0.1, such as 0.2, 0.3, 0.4, 0.5, 0.6, 0.7 or even more, such as in the range of 0.2-0.5, 0.1-0.6, 0.2-0.6, 0.1-0.7, or 0.2-0.7.

In one embodiment user's cumulative current daily credit (current daily credit) and a maximum allowable daily credit for a user (daily credit MAX) are provided, and X is compared to the maximum allowable daily credit for a user (daily credit MAX) with the following formula:

if X+current daily credit>daily credit MAX, then Y=daily credit MAX,

else Y=X+current daily credit,

wherein Y is the credit for the current day, or it is used as a basis for calculating the credit, such as the total credit. The variable Y may be further converted to another unit, for example by using a factor or an algorithm to obtain a desired credit unit.

In one embodiment a change in the average exercise activity units of all users per day (units_{average}) during a predetermined time period is arranged to change the maximum allowable daily credit for a user (daily credit MAX), preferably the units_{average} is directly proportional to the daily credit MAX. For example if the average exercise activity units of all users per day increases during a time period, for example during 1, 2, 3 or 4 weeks, the maximum allowable daily credit for a user may be increased. This way the system may adapt to changing exercise activity of all the users.

In one embodiment the user's exercise activity per day saved in the third party system has been obtained from a user's personal health monitor, such as a wrist device or a mobile terminal, or from a networked exercise device, or from a user exercise tracking platform, or from a combination thereof.

The user credit, which is a calculated value, may be provided as a numerical value, for example a number of monetary units, such as Euros, Dollars and the like. The numerical value may also refer to a virtual or imaginary monetary unit or other unit, for example a unit specific for the virtual environment. The numerical value may also refer to a percentage or to a mark or a point, such as a bonus point, or the like. The stored user credit may be used for several purposes. In one embodiment the user credit is arranged to be exchanged for goods or services, for example in the virtual environment or in a third party system. Therefore, as shown in FIG. 1, the virtual credit may be used in the virtual environment 18 or alternatively it may be used outside the virtual environment as a credit 20 valid in another system. User may choose the format in which the credit is stored and/or where it is stored.

In one embodiment the amount of the stored user credit is arranged to change the properties of the user's personal user interface in the multi-user virtual environment, for example to obtain enhanced properties. The properties of the user interface may include for example the rights of the user, for example rights to do, know or see in the environment, properties of the virtual character representing the user, for example appearance, movement, speed, and the like, the appearance of the user interface, and the like. The more credit the user has the more properties or quality of the properties, in general better or enhanced properties, may be gained.

One embodiment provides a method for converting exercise activity into user credit in a networked multi-user virtual environment, the method comprising
providing the system of any of the preceding claims arranged to run the multi-user virtual environment,
obtaining from a networked third party system information comprising user identification for a user and the user's exercise activity per day, which information has been stored in the third party system,
converting the user's exercise activity into credit for the user, and
storing the user credit in the virtual environment. The converting and other features may be carried out as described above.

One embodiment provides a computer-readable data storage medium having a computer-executable program code stored operative to perform the method described herein. The computer-readable data storage medium may be stored in one or more servers of the virtual environment.

### EXAMPLES

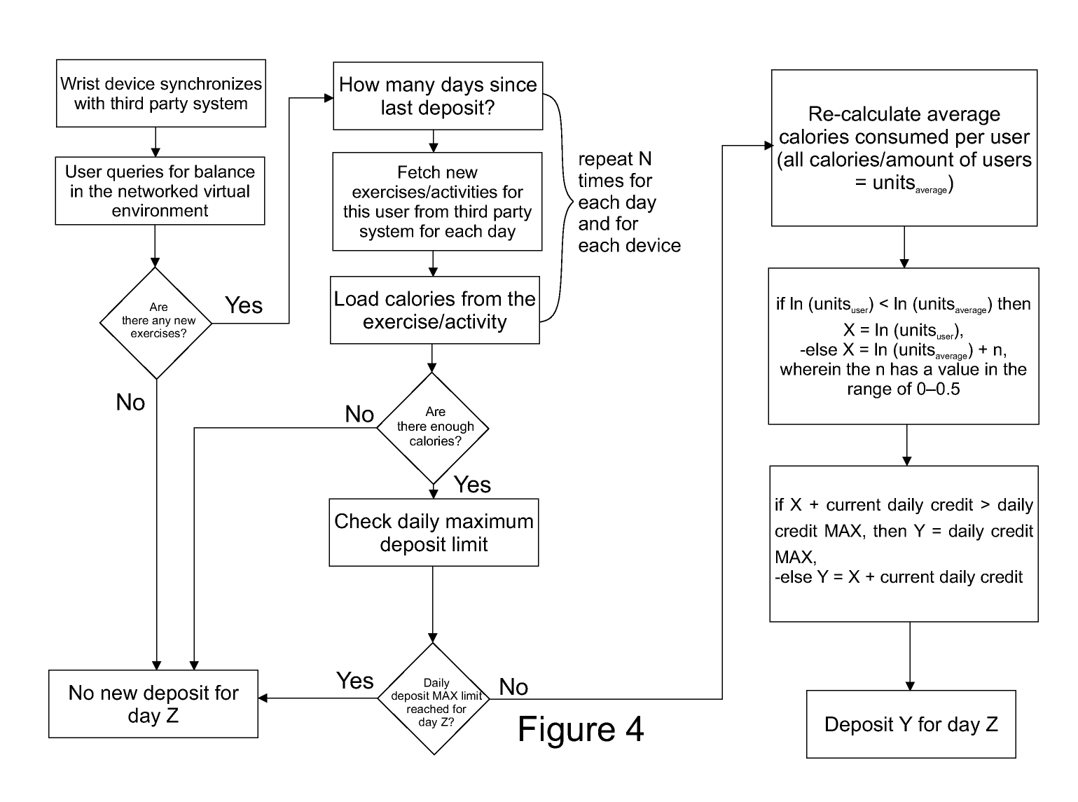

FIG. 4 shows a flowchart describing an example of the actions leading to the conversion of exercise into a deposit of credit in a virtual system account. In the example the user uses a wrist device, which includes a heart rate monitor and accelerometer, and which is arranged to calculate the consumed calories in a time period, more particularly during an exercise event. After training the user starts synchronizing the wrist unit with a networked service, which is a service provided by the wrist unit provider. The user may follow any instructions from the device or from an application run in the device or an external device, such as a mobile terminal. These actions are carried out in an exercise event or immediately after it.

In a separate event the user contacts the networked multi-user virtual environment of the embodiments. If the user has already used the environment before, the user has an account in the environment and the user may log on using a personal user account and a password. If the user has not used the environment before, the user may have to register to the system, and/or to associate a personal user account in the wrist unit provider's system with an account in the networked multi-user virtual environment. The wrist unit provider's system may be called as a partner system or a third party system. Now the two user accounts are associated together in the networked multi-user virtual environment, and the user information gathered in the wrist unit provider's system may be transferred to the networked multi-user virtual environment.

The networked multi-user virtual environment is configured to use OAuth 2 protocol and as a client to contact the partner system. The partner system includes Authorization and Resource servers, which the client communicates with and gets access to the user information therein. The user information including the consumed calories obtained from the wrist device may be retrieved from the partner system to the networked multi-user virtual environment.

A new session is initiated in the networked multi-user virtual environment system. The user makes a query to the system for balance stored in the system. The system checks if there are new exercises stored in the partner system. If there are no new exercises stored, there will be no new deposit for the present day Z. The user may however continue the session in the virtual environment.

If there are new exercises stored in the partner system, the system checks how many days N has been passed since last deposit. The next steps are then repeated N times for each day, and for each device the user may have used, or for each exercise or activity. The system fetches exercises or activities for the user from partner's backend, for example the calories consumed are loaded from an exercise or activity event. The amount of the calories per day is compared to a lower calorie limit per day, which is 150 kcal as shown in FIG. 3. If there are not enough calories, there will be no deposit for the particular day. The amount of the calories per day is compared to a higher calorie limit per day (daily deposit MAX), which is 300 kcal as shown in FIG. 3. If the daily deposit MAX is reached already, there will be no deposit for the particular day. If the daily deposit MAX is not reached, the system proceeds to a next step.

The average calories consumed per user are recalculated as “All calories of all users/Amount of users” to obtain an average calories of all users per day. Then the user's calories per day will be compared with the average calories of all users per day. If the user's calories per day is higher or equal compared to the average calories of all users per day, the user gets higher amount of credit than if the user's calories per day would be lower compared to the average calories units of all users per day. This is calculated by comparing natural logarithms of the user's calories per day (ln(user's calories per day)) and the average calories of all users per day (ln(average calories of all users per day)).

If ln(user's calories per day) is lower than ln(average calories of all users per day), a value X will be ln(user's calories per day). If ln(user's calories per day) is higher than or equal to ln(average calories of all users per day), X will be ln(average calories of all users per day) plus a correction value n, wherein the n has a value in the range of 0-0.5, for example 0.1-0.5. The correction value, as a bonus, is used as an incentive for a user to exercise more.

The above-defined X is then used for determining the user credit for the particular day.

The user's cumulative current daily credit and (current daily credit) a maximum allowable daily credit for a user (daily credit MAX) are provided. X is compared to the maximum allowable daily credit for a user with the following formula:

if X+current daily credit>daily credit MAX, then Y=daily credit MAX,

else Y=X+current daily credit,

wherein Y is the credit for the current day.

Y is then deposited to a user account for the particular day. If there were more than one days N since last deposit, or there were more than one device the user had used, the steps are repeated N times, as mentioned earlier.

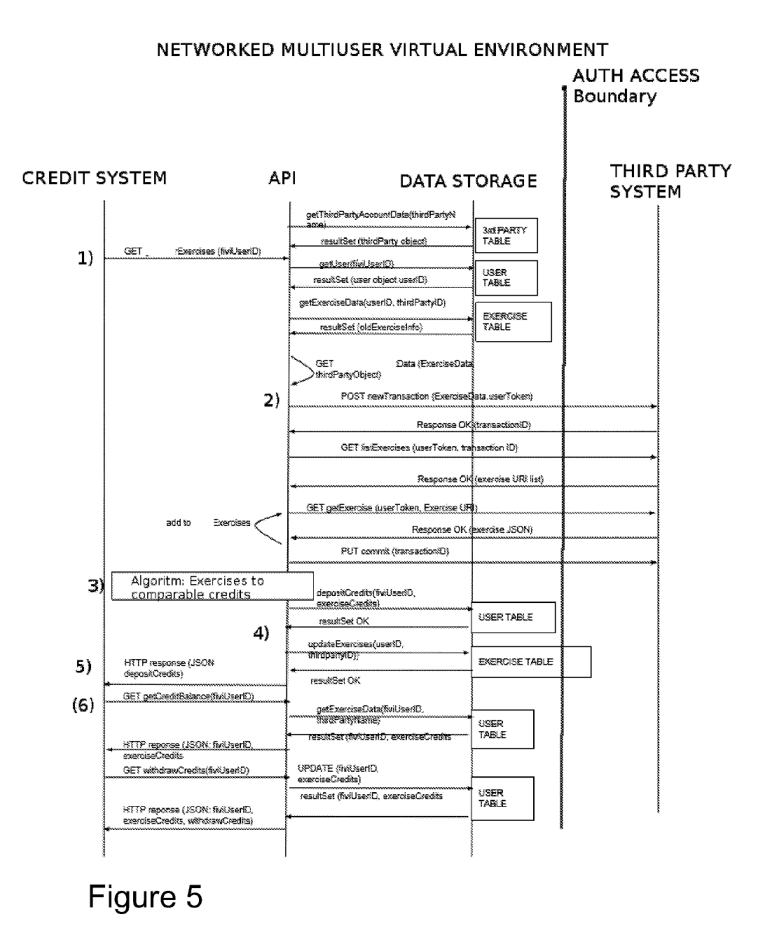

FIG. 5 shows an example of the process wherein the networked multi-user virtual environment includes Credit system, API, and Data storage. The Third party system is separated from the networked multi-user virtual environment by Auth Access Boundary.

When the user wishes to use the exercise activity saved in the third party system, the following will be carried out. The Credit system requests 1), via the API, information from the third party system, which information (3^{rd} party table, user table, and exercise table) has been stored in the Data storage. The Credit system asks the user for permission to use the data from the third party system, which data is owned by the Credit system.

Next a transaction 2) is started with the third party system to fetch the user's exercise data and exercise activity from the third party system. The exercise data is converted 3) into comparable credits by using the algorithm of the embodiments. The credits are stored 4) in the data storage in a desired format, and the algorithm is updated 5). Storing the credit in the desired format means that the algorithm shall be aware of the users choice of format (user setting), into which the credit is converted to during the store procedure and the conversion ratio (system property between formats) used in the process storing. After this the credit information is returned 6) to the user, and the user may freely use the credits.

1. A system for converting exercise activity into user credit (20) in a networked multi-user virtual environment (18), the system comprising one or more networked server(s) arranged to
run the multi-user virtual environment, wherein the system is arranged to
obtain from a networked third party system (16) information comprising user identification for a user and the user's exercise activity, which information is protected data and has been stored in the third party system (16) and wherein the networked multi-user virtual environment (18) has been authorized to access the data,
convert the user's exercise activity into credit (20) for the user in the networked multi-user virtual environment (18), and
store the user credit (20) in the networked multi-user virtual environment (18).

2. The system of claim 1, wherein the cumulative user's exercise activity in exercise activity units per day is compared to a lower limit and/or to a higher limit for a day, wherein the user's exercise activity per day in exercise activity units must be higher than the lower limit and/or lower than the higher limit in order to be accepted and converted into the user credit (20).

3. The system of claim 1, wherein converting the user's exercise activity per day into credit comprises
providing the user's exercise activity per day in exercise activity units (unitsᵤₛₑᵣ), such as calories,
calculating average exercise activity units of all users per day (units_{average}) in the virtual environment by dividing the sum of obtained exercise activity units of all users per day (unitsₐₗₗ) by the number of the users,
comparing the user's exercise activity units per day (unitsᵤₛₑᵣ) with the average exercise activity units of all users per day (units_{average}), wherein
if the user's exercise activity units per day is higher or equal compared to the average exercise activity units of all users per day, the user gets higher amount of credit (20) than if the user's exercise activity units per day is lower compared to the average exercise activity units of all users per day.

4. The system of claim 3, wherein
comparing the user's exercise activity units per day (unitsᵤₛₑᵣ) with the average exercise activity units of all users per day (units_{average}) is carried out by comparing natural logarithms of the user's exercise activity units per day (ln(unitsᵤₛₑᵣ)) and the average exercise activity units of all users per day (ln(units_{average})).

5. The system of claim 4, wherein the comparison is carried out with the following formula:
if ln(unitsᵤₛₑᵣ)<ln(units_{average}) then X=ln(unitsᵤₛₑᵣ),
else X=ln(units_{average})+n,
for example wherein the n has a value in the range of 0-0.5, preferably 0.1-0.5, wherein X is used for determining the user credit (20) for the current day.

6. The system of claim 5, wherein user's cumulative current daily credit (current daily credit) and a maximum allowable daily credit for a user (daily credit MAX) are provided, and X is compared to the maximum allowable daily credit for a user (daily credit MAX) with the following formula:
if X+current daily credit>daily credit MAX, then Y=daily credit MAX,
else Y=X+current daily credit,
wherein
Y is the total for the current day, or it is used as a basis for calculating the credit.

7. The system of claim 6, wherein a change in the average exercise activity units of all users per day (units_{average}) during a predetermined time period is arranged to change the maximum allowable daily credit for a user (daily credit MAX).

8. The system of claim 1, wherein the user's exercise activity per day saved in the third party system (16) has been obtained from a user's personal health monitor, such as a wrist device or a mobile terminal, or from a networked exercise device, or from a user exercise tracking platform, or from a combination thereof (10, 12, 16).

9. The system of claim 1, wherein the user credit (20) is arranged to be exchanged for goods or services, for example in the virtual environment (18) or in a third party system (16).

10. The system of claim 1, wherein the amount of the stored user credit (20) is arranged to change the properties of the user's personal user interface in the multi-user virtual environment (18).

11. A method for converting exercise activity into user credit (20) in a networked multi-user virtual environment (18), the method comprising
providing the system of claim 1 arranged to run the multi-user virtual environment,
obtaining from a networked third party system (16) information comprising user identification for a user and the user's exercise activity per day, which information is protected data and has been stored in the third party system and wherein the networked multi-user virtual environment (18) has been authorized to access the data,
converting the user's exercise activity into credit (20) for the user in the networked multi-user virtual environment (18), and
storing the user credit (20) in the networked multi-user virtual environment (18).

12. The method of claim 11, comprising comparing the cumulative user's exercise activity in exercise activity units per day to a lower limit and/or to a higher limit for a day, wherein the user's exercise activity per day in exercise activity units must be higher than the lower limit and/or lower than the higher limit in order to be accepted and converted into the user credit (20).

13. The method of claim 11, wherein converting the user's exercise activity per day into credit comprises
providing the user's exercise activity per day in exercise activity units (unitsᵤₛₑᵣ), such as calories,
calculating average exercise activity units of all users per day (units_{average}) in the virtual environment by dividing the sum of obtained exercise activity units of all users per day (unitsₐₗₗ) by the number of the users,
comparing the user's exercise activity units per day (unitsᵤₛₑᵣ) with the average exercise activity units of all users per day (units_{average}), wherein
if the user's exercise activity units per day is higher or equal compared to the average exercise activity units of all users per day, the user gets higher amount of credit (20) than if the user's exercise activity units per day is lower compared to the average exercise activity units of all users per day.

14. The method of claim 13, wherein
comparing the user's exercise activity units per day (unitsᵤₛₑᵣ) with the average exercise activity units of all users per day (units_{average}) is carried out by comparing natural logarithms of the user's exercise activity units per day (ln(unitsᵤₛₑᵣ)) and the average exercise activity units of all users per day (ln(units_{average})).

15. The method of claim 14, wherein the comparison is carried out with the following formula:
if ln(unitsᵤₛₑᵣ)<ln(units_{average}) then X=ln(unitsᵤₛₑᵣ),
else X=ln(units_{average})+n,
for example wherein the n has a value in the range of 0-0.5, preferably 0.1-0.5, wherein X is used for determining the user credit (20) for the current day.

16. The method of claim 15, wherein user's cumulative current daily credit (current daily credit) and a maximum allowable daily credit for a user (daily credit MAX) are provided, and X is compared to the maximum allowable daily credit for a user (daily credit MAX) with the following formula:
if X+current daily credit>daily credit MAX, then Y=daily credit MAX,
else Y=X+current daily credit,
wherein
Y is the total for the current day, or it is used as a basis for calculating the credit.

17. The method of claim 16, wherein a change in the average exercise activity units of all users per day (units_{average}) during a predetermined time period is arranged to change the maximum allowable daily credit for a user (daily credit MAX).

18. The method of claim 11, wherein the user's exercise activity per day saved in the third party system (16) has been obtained from a user's personal health monitor, such as a wrist device or a mobile terminal, or from a networked exercise device, or from a user exercise tracking platform, or from a combination thereof (10, 12, 16).

19. The method of claim 11, wherein the user credit (20) is exchanged for goods or services, for example in the virtual environment (18) or in a third party system (16).

20. The method of claim 11, wherein the amount of the stored user credit (20) is arranged to change the properties of the user's personal user interface in the multi-user virtual environment (18).

21. A computer-readable data storage medium having a computer-executable program code stored operative to perform the method of claim 11.
